(19) United States
(12) Patent Application Publication
(10) Pub. No.: US 2024/0181111 A1
(43) Pub. Date: Jun. 6, 2024

(54) SYSTEMS AND METHODS FOR AIR SANITIZATION USING FORMULAS CONTAINING AN ORGANIC ACID

(71) Applicant: The Clorox Company, Oakland, CA (US)

(72) Inventors: Yintao Sun, Union City, CA (US); Xuanrong Guo, Palo Alto, CA (US); Thomas Fahlen, San Jose, CA (US); Mark Pszczolkowski, Vallejo, CA (US); Marjan M. Hashemi, San Ramon, CA (US); Fatima A. Hussain, Dublin, CA (US)

(21) Appl. No.: 18/528,419

(22) Filed: Dec. 4, 2023

Related U.S. Application Data
(60) Provisional application No. 63/386,268, filed on Dec. 6, 2022.

(51) Int. Cl. A61L 9/14; A01N 31/02; A01N 37/00; A01P 1/00

(52) U.S. Cl. CPC A61L 9/14; A01N 31/02; A01N 37/00; A01P 1/00; A61L 2101/36

(57) ABSTRACT

Systems and methods for air sanitization. The system includes a device configured to create aerosolized droplets of an antimicrobial formulation with an average particle size sufficiently small so that the formulation provides at least a 3-log reduction against Staphylococcus aureus or another target microbe within 60 minutes. The antimicrobial formulation includes one or more organic acids (e.g., lactic acid) and optionally one or more diols (e.g., 1,2-hexanediol). Where both are included, the organic acid(s) and diol(s) may have a vapor pressure of at least 0.02 Pa. Where just the organic acid is included, the organic acid has a vapor pressure of at least 0.10 Pa. The formulation may further include a buffer, a surfactant, water, a fragrance, etc. The formulation may be delivered as an aerosol, using a pump sprayer, a mister, a nebulizer, a vaporizer, a cold fogger, a thermal fogger, an ultrasonic dispensing device, an electrostatic sprayer, etc.

### CROSS-REFERENCE TO RELATED APPLICATIONS

The present application claims the benefit of U.S. Provisional Patent Application Ser. No. 63/386,268, filed Dec. 6, 2022, entitled “SYSTEMS AND METHODS FOR AIR SANITIZATION USING FORMULAS CONTAINING AN ORGANIC ACID” the disclosure of which is incorporated by reference in its entirety.

### BACKGROUND OF THE INVENTION

### 1. The Field of the Invention

The present invention relates to air sanitization systems for use against airborne bacteria and/or viruses. Such systems may include a formulation for delivery through any of various mechanisms, including but not limited to aerosols, misters, pump sprayers, HVLP sprayers, ULV foggers, thermal foggers, electrostatic sprayers, and/or ultrasonic dispensing devices.

### 2. Description of Related Art

Sanitization sprays are commercially available, however, most of these sprays are meant to be sprayed to provide sanitization of various surfaces. Examples of such include CLOROX DISINFECTING MIST and LYSOL DISINFECTANT SPRAY. Disinfectant foggers are also available which are capable of airborne disinfection, however, these devices are often complex and expensive, and can result in long exposure to toxic chemicals. While sanitization sprays and disinfectant foggers have shown some success in providing sanitization or disinfection, there is a continuing need for improved systems that would provide for sanitization or disinfection against airborne bacteria and viruses in a safe, effective, and economical manner.

### BRIEF SUMMARY

This disclosure relates to systems for use in air sanitization or disinfection, as well as related methods for providing air sanitization or disinfection. Current sanitization formulations are typically directed towards surface disinfection. However, there is a growing need to reduce viruses and bacteria while such are still airborne instead of waiting for such pathogens to reach a surface or come in contact with an animal or human host. When developing a new sanitization or disinfection formulation and system, it is helpful to take various factors into account. For example, in an embodiment, the formulation would only include components which are identified in the EPA's safer chemical ingredient list as “green circle” components (e.g., which qualify for the EPA's DfE program). In addition, in an embodiment, active components of the formulation should have a minimum threshold vapor pressure, in order to stay airborne for a sufficient period of time to provide the desired sanitization or disinfection. Additionally, in an embodiment, the formulation would have a European Chemicals Association (ECHA) or similar regulatory agency long term inhalation threshold which is acceptable for the general population. Additionally the formulation would of course need to provide significant antimicrobial properties (e.g., a significant log reduction in a target microbe, for example a 3-log reduction) while airborne. Additionally, the formulation may be aerosolized into droplets using a device that creates droplets with an average particle size of less than about 200 μm, less than about 150 μm, or less than about 100 μm. Finally, consumer preferences may be accommodated, such as inclusion of a fragrance, case of use, and non-toxicity. Embodiments of the present formulations may provide for antimicrobial efficacy against bacteria, viruses, and/or fungi present in the air as well as on hard, soft, and nonporous surfaces.

In one aspect, the present invention is directed to a system for air sanitization including a device configured to create aerosolized droplets of an antimicrobial formulation with an average particle size that is sufficiently small to allow airborne sanitization (e.g., less than about 200 μm, less than about 150 μm, less than about 100 μm such as from about 5 μm to about 60 μm), where the antimicrobial formulation includes one or more organic acids. In some embodiments, one or more diols may also be present. In an embodiment, the organic acid has a vapor pressure under standard conditions (20° C. and 1 atm) of at least 0.1 Pa, at least 0.15 Pa, at least 0.2 Pa, at least 0.25 Pa, at least 0.3 Pa, at least 0.35 Pa or at least 0.40 Pa, such as from 0.10 Pa to about 300 Pa. Where both one or more organic acids and the one or more diols are present, the organic acids and the diols may be selected to specifically have a vapor pressure of at least 0.02 Pa at room temperature (e.g., standard conditions). Related methods of use, for such a system are also contemplated. Any components (such as such organic acids and/or diols) or other components present in a significant fraction (e.g., greater than 0.1%, greater than 0.2%, greater than 0.5%, or greater than 1%) may have an ECHA or similar long term inhalation threshold limit for the general population of at least 10 mg/m³ or at least 20 mg/m³.

In an embodiment, an exemplary organic acid providing such characteristics includes lactic acid, while an exemplary diol may be 1,2-hexanediol. In an embodiment, the diols may consist of 1,2-hexanediol (rather than a mixture of different diols).

In an embodiment, the antimicrobial formulation may contain from 0.1% to 5% (e.g., about 2%) lactic acid.

In an embodiment, the antimicrobial formulation may contain from 0.5% to 10% (e.g., about 4-5%) of 1,2-hexanediol.

In an embodiment, the pH of the antimicrobial formulation may be from about 1.5 to about 6, from about 2 to about 5, from about 2 to about 4, from about 2 to about 3, from about 2 to about 2.5, or from about 2.0 to about 2.4.

In an embodiment, the diol and organic acid may be included within specific ratios relative to one another (e.g., 3:1 to 1:3). In an embodiment, the diol is included in a greater concentration than the organic acid. For example, the concentration ratio of the diol (e.g., 1,2-hexanediol) to the organic acid (e.g., lactic acid) may be from about 1:1 to about 4:1, from about 1.2:1 to about 4:1, from about 1.5:1 to about 3.5:1, or from about 1.5:1 to about 2.5:1. In another embodiment, lactic acid may dominate, e.g., with a ratio of lactic acid to diol of greater than 1, such as from about 1.1 to about 2:1, or from about 1:1 to about 1.5:1, or from about 1:1 to about 1.3:1 (e.g., about 1.1). The particular concentrations and/or ratios of the 1,2-hexanediol or other diol to the lactic acid or other organic acid may be an important lever, even critical in some embodiments, to achieving the desired air sanitization. The average particle size provided from the dispensing device (e.g., less than about 200 μm, less than 150 μm, less than 100 μm, less than 80 μm, less than about 60 μm, such as from about 5 μm to about 60 μm) may also be an important, even critical characteristic.

In an embodiment, the antimicrobial formulation may include a buffer such as citric acid, e.g., present from about 0.1% to about 5%.

In an embodiment, the antimicrobial formulation may further include a surfactant and/or fragrance.

In an embodiment, the antimicrobial formulation may provide at least a 3, 4, or 5 log reduction (e.g., at least 3.0 log reduction) against an airborne target virus or other microbe (e.g., Staphylococcus aureus) within 60 minutes or less (e.g., sometimes even within 30 minutes, or within 10 minutes).

In an embodiment, the formulation may be void or substantially void of propylene glycol, dipropylene glycol, triethylene glycol, hexylene glycol, other glycols including ether groups, hypochlorite, ethanol, isopropanol, 1-propanol, 1-hexanol and/or resorcinols.

In another aspect, the system includes a device configured to create aerosolized droplets of the antimicrobial formulation with a sufficiently small average particle size (e.g., less than about 200 μm, less than about 150 μm, less than about 100 μm, less than about 80 μm, less than about about 60 μm, such as from about 5 μm to about 60 μm), where the formulation includes about 0.1% to about 5% by weight of at least one of lactic acid or citric acid, about 0.5% to about 10% by weight of 1,2-hexanediol, at least one of a fragrance or surfactant, and at least 80% by weight water. In an embodiment, both the lactic acid and the citric acid may be present in a range from about 0.1% to about 5% by weight. In such an embodiment, the antimicrobial formulation may have a pH from 2 to 4 and may provide at least a 3-log reduction against an airborne target microbe within 60 minutes.

In another aspect, the system includes a device configured to create aerosolized droplets of the antimicrobial formulation with a relatively small average particle size as described herein, where the formulation includes about 0.1% to about 5% by weight of lactic acid, about 0.5% to about 10% by weight of 1,2-hexanediol, optionally about 0.1% to about 5% by weight of citric acid, a fragrance, surfactant, or another desired adjunct, and at least 80% by weight water. In such an embodiment, the antimicrobial formulation may have a pH from about 2 to about 4, and may provide at least a 3-log reduction against an airborne target microbe (e.g., Staphylococcus aureus) within 60 minutes or less.

Another aspect is directed to a method for providing air sanitization treatment, where the method includes providing an antimicrobial formulation, e.g., including one or more organic acids, such as about 0.1% to about 5% by weight of lactic acid, optionally a diol, such as about 0.5% to 10% by weight of 1,2-hexanediol, and at least 80% water by weight, where the formulation has a pH as described herein, e.g., from about 2 to about 4, provides at least a 3 log reduction against an airborne target microbe within 60 minutes or less, and is delivered as a mist to the air to be sanitized. The formulation may be delivered using a mister, a pump sprayer, a nebulizer, a vaporizer, a cold fogger, a thermal fogger, an ultrasonic dispensing device (e.g., ultrasonic diffuser), an electrostatic sprayer, combinations thereof, or other delivery methods. In an embodiment, the formulation may be a 2-part formulation, where delivery of the antimicrobial formulation is stepwise where any diol (e.g., the 1,2-hexanediol) is delivered first and the lactic acid or other organic acid is delivered second.

In an embodiment, a total number of moles of the lactic acid or other organic acid and the 1,2-hexanediol or other diol delivered to the air to be sanitized is from about 0.1 to about 4 mmol/m³, or from about 0.1 to about 2 mmol/m³. The mmol/m³ values can be calculated based on the assumption that all chemistries are fully vaporized into the gaseous form. Such calculated values may therefore be an overestimation relative to empirical values. The empirical values for the total number of moles of the lactic acid or other organic acid and the 1,2-hexanediol or other diol delivered to the air to be sanitized may be e.g., from about 0.05 to about 1.5 mmol/m³.

In an embodiment, the selected organic acid has a vapor pressure ranging from 0.10 Pa to about 300 Pa, or from 0.10 Pa to about 200 Pa, from 0.40 Pa to about 300 Pa, from 0.40 Pa to about 200 Pa, from 0.41 Pa to about 172 Pa, or from about 5 Pa to about 300 Pa, or from about 5 Pa to about 200 Pa under standard conditions (room temperature and 1 atm).

In an embodiment, the selected organic acid has an aqueous solubility of at least about 10 g, at least about 20 g, at least about 24.99 g, such as from about 10 g to about 100 g, about 10 g to about 50 g, or about 20 g to about 50 g of the organic acid per 100 grams of water under standard conditions (room temperature and 1 atm).

Further features and advantages of the present invention will become apparent to those of ordinary skill in the art in view of the detailed description of preferred embodiments below.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings.

FIG. 1 charts the log reduction of prototype #1 (4.25% 1,2-hexanediol, 2% Lactic acid) against airborne Staphylococcus aureus over time using different dosages. Log reduction is normalized against untreated baseline.

FIG. 2 charts the log reduction of prototype #1 (4.25% 1,2-hexanediol, 2% Lactic acid) against airborne MS2 over time using different dosages. Log reduction is normalized against untreated baseline.

FIG. 3 charts the normalized log reduction against phi 6 (log reduction per g/m³ of formula delivered) versus 1,2-hexanediol mole fraction versus molar concentration of formula delivered (mmol/m³) into a treatment space at 10 minute exposure time for various formulations including lactic acid and 1,2-hexanediol. Testing was performed in a 1 m³ air chamber delivered via a sprayer, with a Dv50 of about 70 μm.

FIG. 4 charts the log reduction against phi 6 at different 1,2-hexanediol and lactic acid mole fraction ratios for a 60 minute exposure time. Total concentration of 1,2-hexanediol and lactic acid are kept constant at 254 mmol, 582 mmol, and 1164 mmol, and the log reduction is normalized against an untreated baseline. Testing was performed in a 1 m³ air chamber where 1 g of chemistry is delivered via a sprayer, with a Dv50 of about 70 μm.

FIG. 5 illustrates the log reduction against phi 6 at 3.5% 1,2-hexanediol and varying levels of lactic acid. Log reduction was normalized against untreated baseline.

FIG. 6 charts the log reduction of prototype #1 (4.25% 1,2-hexanediol, 2% Lactic acid), and prototypes containing an equal amount of lactic acid in acid form and 4.25% HDO after adjustment of pH, with and without citric acid buffer, over time. Log reduction is normalized against untreated baseline. Testing was performed in a 1 m³ air chamber delivered via a sprayer, with a Dv50 of about 70 μm.

FIG. 7 charts the log reduction (log reduction/g of formulation delivered) of prototype #1 (4.25% 1,2-hexanediol, 2% Lactic acid) over time for various methods of delivery, including stepwise release 1 (HDO first), stepwise release 2 (LA first), and separate but simultaneous release of the lactic acid and 1,2-hexanediol components of the antimicrobial formulation. Log reduction is normalized against untreated baseline. Stepwise release included release of one chemistry first, followed by delivery of the second chemistry after 2 minutes. Separate release included simultaneous release of both chemistries from different reservoirs, at different locations within the enclosed volume. Testing was performed in a 1 m³ air chamber delivered via a sprayer, with a Dv50 of about 70 μm.

FIG. 8 charts the log reduction of prototype #1 (4.25% 1,2-hexanediol, 2% Lactic acid) against Staphylococcus aureus based on the particle size using a variety of sprays. Testing was performed in a 1 m³ air chamber.

FIG. 9 charts the log reduction of prototype #1 (4.25% 1,2-hexanediol, 2% Lactic acid) against Staphylococcus aureus and Klebsiella pneumoniae on a soft cotton surface with a 5 minute exposure time.

FIG. 10 charts the log reduction against airborne phi 6 for various formulas including an organic acid alone (e.g., lactic acid), a diol alone (e.g., 1,2-hexanediol), and both the organic acid and diol together, showing synergistic results associated with inclusion of both the organic acid and the diol. Testing was performed in a 1 m³ air chamber delivered via a sprayer, with a Dv50 of about 70 μm. The illustrated log reductions were charted against an untreated baseline.

FIG. 11 charts the log reduction against airborne phi 6 for various organic acids, tested at various vapor pressures of such organic acid. Testing was performed in a 1 m³ air chamber delivered via a sprayer, with a Dv50 of about 70 μm. For each test, 2 g of 222 mmol organic acid was delivered. The illustrated log reductions at 15 minutes contact time were charted against an untreated baseline. A variety of organic acids show at least a 3-log reduction at vapor pressures from 0.41 Pa to 172 Pa.

### DETAILED DESCRIPTION OF THE PREFERRED EMBODIMENTS

### I. Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising” which is synonymous with “including,” “containing,” or “characterized by,” is inclusive or open-ended and does not exclude additional, unrecited elements or method steps.

The term “consisting essentially of” limits the scope of a claim to the specified materials or steps “and those that do not materially affect the basic and novel characteristic(s)” of the claimed invention.

The term “consisting of” as used herein, excludes any element, step, or ingredient not specified in the claim.

It must be noted that, as used in this specification and the appended claims, the singular forms “a,” “an” and “the” include plural referents unless the content clearly dictates otherwise. Thus, for example, reference to a “surfactant” includes one, two or more surfactants.

Unless otherwise stated, all percentages, ratios, parts, and amounts used and described herein are by weight.

Numbers, percentages, ratios, or other values stated herein may include that value, and also other values that are about or approximately the stated value, as would be appreciated by one of ordinary skill in the art. As such, all values herein are understood to be modified by the term “about”. Such values thus include an amount or state close to the stated amount or state that still performs a desired function or achieves a desired result. A stated value should therefore be interpreted broadly enough to encompass values that are at least close enough to the stated value to perform a desired function or achieve a desired result, and/or values that round to the stated value. The stated values include at least the variation to be expected in a typical manufacturing or other process, and may include values that are within 10%, within 5%, within 1%, etc. of a stated value.

Some ranges may be disclosed herein. Additional ranges may be defined between any values disclosed herein as being exemplary of a particular parameter. All such ranges are contemplated and within the scope of the present disclosure.

| As used herein, the term “between” is inclusive of any endpoints noted relative to a described range.

In the application, effective amounts are generally those amounts listed as the ranges or levels of ingredients in the descriptions, which follow hereto. Unless otherwise stated, amounts listed in percentage (“%'s”) are in weight percent (based on 100% active) of any composition.

The phrase ‘free of’ or similar phrases if used herein means that the composition or article comprises 0% of the stated component, that is, the component has not been intentionally added. However, it will be appreciated that such components may incidentally form thereafter, under some circumstances, or such component may be incidentally present, e.g., as an incidental contaminant.

The phrase ‘substantially free of’ or similar phrases as used herein means that the composition or article preferably comprises 0% of the stated component, although it will be appreciated that very small concentrations may possibly be present, e.g., through incidental formation, contamination, or even by intentional addition. Such components may be present, if at all, in amounts of less than 1%, less than 0.5%, less than 0.25%, less than 0.1%, less than 0.05%, less than 0.01%, less than 0.005%, less than 0.001%, or less than 0.0001%. In some embodiments, the compositions or articles described herein may be free or substantially free from any specific components not mentioned within this specification.

The antimicrobial formulation as described herein may provide sanitization, disinfection, or sterilization, other cleaning, or other treatment. As used herein, the term “sanitize” shall mean the reduction of “target” contaminants in the inanimate environment to levels of at least 3 logs below the untreated condition, or that reduces a “target” bacterial population by significant numbers where public health requirements have not been established. By way of example, an at least 99% reduction in bacterial population within a 24-hour time period is deemed “significant.” Greater levels of reduction (e.g., 99.9%, 99.99%, etc.) are possible, as are faster treatment times (e.g., within 10 minutes, within 5 minutes, within 4 minutes, within 3 minutes, within 2 minutes, within 1 minute, or within 30 seconds), when sanitizing or disinfecting.

As used herein, the term “disinfect” shall mean the elimination of at least 6 logs of many or all “target” pathogenic microorganisms on surfaces with the exception of bacterial endospores.

As used herein, the term “sterilize” shall mean the complete elimination or destruction of all forms of “target” microbial life and which is authorized under the applicable regulatory laws to make legal claims as a “sterilant” or to have sterilizing properties or qualities.

Some embodiments may provide for at least a 2 or more log reduction (e.g., 3-log reduction, 4-log reduction, 5-log reduction, or 6-log reduction) in a bacterial population within a designated time period (e.g., 10 minutes, 5 minutes, 4 minutes, 3 minutes, 2 minutes, 1 minute, 30 seconds, or the like) relative to a baseline. A 2-log reduction is equivalent to a 99% reduction, a 3-log reduction is equivalent to at least a 99.9% reduction, a 4-log reduction is equivalent to at least a 99.99% reduction, a 5-log reduction is equivalent to at least a 99.999% reduction, etc. An example of a target microbe may be Staphylococcus aureus. It will be appreciated that antimicrobial efficacy can also be achieved against other target microbes, numerous examples of which will be apparent to those of skill in the art.

The term “Design for the Environment” or “DfE” means the U.S. EPA program that is focused on identifying safer sanitizing and disinfecting active ingredients. The EPA has a special approval process for products that met the DfE criteria. The EPA, as part of the DfE program has identified certain active ingredients that are approved for antimicrobial cleaning products and authorized to use the DfE logo. The antimicrobial cleaning products that have been approved under the DfE program may be found under https://www.epa.gov/pesticide-labels/design-environment-logo-antimicrobial-pesticide-products #authorizeddfe. All products approved for DfE program must have ingredients that meet the “Safer Choice Standard” according to https://www.epa.gov/pesticide-labels/design-environment-logo-antimicrobial-pesticide-products #approved. In an embodiment, the present composition could be formulated to meet DE guidelines, or similar regulatory guidelines.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

### II. Exemplary Air Sanitization Systems and Formulas

In an aspect, the present invention is directed to systems that include an antimicrobial formulation to be used in air sanitization treatments. Additionally, the present disclosure is directed to methods for providing such treatments. To be effective in air sanitization, an antimicrobial formulation should have acrosolized antimicrobial formula droplets where the antimicrobial molecules are capable of leaving the aerosolized droplets (due to droplet size and their vapor pressure) and affecting the airborne microbial target. The microbial target may include both enveloped viruses (e.g., phi 6, SARS-Covid surrogates) and non-enveloped viruses (e.g., MS2), as well as other pathogenic bacteria or fungi (e.g., S. aureus, A. niger, Klebsiella pneumoniae, Pseudomonas aeruginosa, etc.).

### A. Organic Acids and Diols

Embodiments of the antimicrobial formulation include at least one organic acid and optionally at least one diol. In an embodiment, the organic acid(s) have a vapor pressure in a range from 0.10 Pa to about 300 Pa at room temperature (e.g., 20° C.), standard conditions (e.g., 1 atm). Where both an organic acid and a diol is included, the organic acid(s) and diol(s) in the antimicrobial formulation may have a vapor pressure of at least 0.02 Pa at room temperature standard conditions. In an embodiment, the organic acid(s), any diol(s), as well as any other components present in relatively higher concentrations may have a European Chemicals Agency (ECHA) long term inhalation threshold limit for the general population of at least 10 mg/m³. It will be apparent that various components included in very small amounts (e.g., not greater than 1%, not greater than 0.5%, or not greater than 0.1%) may not need to meet such a limit.

In an embodiment, the organic acid is a short chain organic acid, e.g., having no more than about 12, no more than about 10, no more than about 8, no more than about 6, or no more than about 4 carbon atoms. In an embodiment, the organic acid is a mono-organic acid (e.g., as opposed to a dicarboxylic or polycarboxylic acid). In an embodiment, where included, the diol is a short chain diol, e.g., having no more than about 16, no more than about 12, no more than about 10, no more than about 8, no more than about 6, or no more than about 4 carbon atoms. In an embodiment, the organic acid(s) is or includes lactic acid and the diol(s) is or includes 1,2-hexanediol. The lactic acid or other organic acid(s) may be included from about 0.05%, from about 0.1%, from about 0.5%, from about 1%, from about 1.5%, from about 2%, up to about 10%, up to about 8%, up to up to about 6%, up to about 5%, up to about 4%, or up to about 3% by weight of the antimicrobial formulation. In an embodiment, the lactic acid is included at about 2% by weight of the antimicrobial formulation. The 1,2-hexanediol or other diol(s) may be included from about 0.05%, from about 0.1%, from about 0.5%, from about 1%, from about 2%, from about 3%, from about 4%, up to about 15%, up to about 12%, up to about 10%, up to about 8%, up to about 7%, up to about 6%, or up to about 5% by weight of the antimicrobial formulation. In an embodiment, the 1,2-hexanediol or other diol(s) are included at about 4.25% by weight of the antimicrobial formulation. It will be appreciated that concentrates may also be possible, e.g., provided with lactic acid, other organic acid, and/or 1,2-hexanediol or other diol concentrations of up to about 70%, up to about 60%, up to about 50%, up to about 40%, up to about 30%, or up to about 20%, e.g., which would be diluted down, at the time of delivery, e.g., to the more dilute values noted above.

In an embodiment, the concentration ratio of the 1,2-hexanediol or other diol to the lactic acid or other organic acid is from about 3:1 to about 1:3, or in an embodiment, may be greater than 1:1, such as from about 1.2:1 to about 4:1, or from about 1.5:1 to about 3.5:1, or from about 1.5:1 to about 2.5:1 (e.g., about 2:1). In another embodiment, lactic acid may dominate, e.g., with a ratio of lactic acid to diol of greater than 1, such as from about 1.1 to about 2:1, or from about 1:1 to about 1.5:1, or from about 1:1 to about 1.3:1 (e.g., about 1.1). Such concentrations, ratios, and the airborne average droplet particle sizes provided by the present systems may be important, even critical characteristics to achieving the air sanitization characteristics described herein.

In an embodiment, two or more diols may be provided. Similarly, two or more organic acids may be provided. In another embodiment, only a single diol is provided (e.g., the diol consists of 1,2-hexanediol). In an embodiment, only a single organic acid is provided (e.g., the organic acid consists of lactic acid). Non-limiting examples of organic acids include maleic acid, methanesulfonic acid, benzoic acid, levulinic acid, glycolic acid, lactic acid, pyruvic acid, propionic acid, and/or acetic acid. Organic acids exhibiting a vapor pressure within a range of 0.10 Pa to about 300 Pa, or 0.40 Pa to 200 Pa under standard conditions may be particularly desirable. Examples of such include, but may not be limited to glycolic acid, lactic acid, and pyruvic acid.

In another aspect, the pH of the antimicrobial formulation is kept low. A low pH can be important for lactic acid or other acids to achieve air sanitization efficacy. Lactic acid is most efficacious in the acid form compared to the conjugate base form (lactate salts). For example, the acid form of lactic acid is more volatile. At a pH of 2.2, at least 95% of present lactic acid is in the acidic form (lactic acid has a pKₐ of 3.79). While lactic acid has higher efficacy at low pH, a neutral pH formulation has better aesthetics and potentially lower toxicity. Therefore, a pH that provides a balance between microefficacy of lactic acid and aesthetics and toxicity levels is desirable.

By way of example, the pH may be at least about 1, at least about 1.5, at least about 2, up to about 6.5, up to about 6, up to about 5, up to about 4, up to about 3, up to about 2, up to about 2.5, or up to about 2.4. In an embodiment, the pH is kept between about 2 and about 4, or about 2 and about 3. The pH may be maintained by the addition of a buffer. In some embodiments, the buffer includes citric acid. It is important to note that the citric acid is present as a buffer, rather than for any primary antimicrobial benefit. The citric acid buffer may be included in an amount of at least about 0.05%, at least about 0.1%, up to about 6%, up to about 5%, up to about 4%, up to about 3%, up to about 2%, up to about 1.5%, or up to about 1% by weight of the antimicrobial formulation.

### B. Solvents

In an embodiment, the formulation may be free, or substantially free of additional solvents, other than the diol. That said, in other embodiments, a solvent may be present, such as a glycol ether solvent. Exemplary glycol ether solvents include, but are not limited to, ethylene glycol monopropyl ether, ethylene glycol monobutyl ether, ethylene glycol monohexyl ether, ethylene glycol phenyl ether, propylene glycol n-propyl ether, propylene glycol monobutyl ether, propylene glycol t-butyl ether, diethylene glycol monocthyl or monopropyl or monobutyl ether, di- or tri-polypropylene glycol methyl or ethyl or propyl or butyl ether, acetate and/or propionate esters of glycol ethers. A glycol ether or other solvent may be included from about 0.1%, from about 0.25%, up to about 5%, up to about 4%, up to about 3%, up to about 2%, or up to about 1% by weight of the formulation. Other solvents, surfactants, and various other adjuvants often included in sanitization or disinfection formulations may optionally be present. While some embodiments may include lower alcohol solvents (e.g., C₁-C₄ mono-alcohols), the amount of such volatile solvents may be limited, e.g., to less than about 50%, less than about 40%, less than about 30%, less than about 20%, less than about 10%, less than about 5%, less than about 3%, less than about 2%, less than about 1%, less than about 0.5%, or less than about 0.3% by weight. In some embodiments, the composition may be free of, or substantially free of, such lower mono-alcohol or other highly volatile solvents.

In an embodiment, the antimicrobial formulation may be void or substantially void of glycols other than hexanediol or even glycol ethers. Examples of such glycols include, but are not limited to propylene glycol, dipropylene glycol, triethylene glycol, and hexylene glycol. In an embodiment, the composition may similarly be free, or substantially free of resorcinols.

### C. Surfactants

In an embodiment, the antimicrobial formulation may include one or more surfactants (e.g., particularly an anionic surfactant and/or a nonionic surfactant). In some embodiments, one or more surfactants may be included in an amount of at least about 0.025%, at least about 0.05%, at least about 0.1%, at least about 0.2%, at least about 0.3%, at least about 0.4%, at least about 0.5%, up to about 10%, up to about 3%, up to about 2%, or up to about 1%, by weight of the antimicrobial formulation.

Those of skill in the art will appreciate that any among a wide variety of surfactants (e.g., anionic, cationic, non-ionic, zwitterionic, and/or amphoteric) may be included in the formulation, as desired. Where included, a surfactant may be present from about 0.05%, from about 0.1%, up to about 10%, up to about 5%, up to about 4%, up to about 3%, up to about 2%, or up to about 1% by weight of the formulation. Various surfactants and other optional adjuvants are disclosed in U.S. Pat. No. 3,929,678 to Laughlin and Heuring, U.S. Pat. No. 4,259,217 to Murphy, U.S. Pat. No. 5,776,872 to Giret et al.; U.S. Pat. No. 5,883,059 to Furman et al.; U.S. Pat. No. 5,883,062 to Addison et al.; U.S. Pat. No. 5,906,973 to Ouzounis et al.; U.S. Pat. No. 4,565,647 to Llenado, and U.S. Publication No. 2013/0028990. The above patents and applications are each herein incorporated by reference in their entirety.

Examples of nonionic surfactants include, but are not limited to, alcohol ethoxylates, alcohol propoxylates, other alcohol alkoxylates including fatty (e.g., C₆, C₈, C₁₀, or C₁₂, or higher) alcohols or other constituents that have been alkoxylated to include both ethoxy and propoxy groups (EO-PO surfactants), alkyl phosphine oxides, alkyl glucosides and alkyl pentosides, alkyl glycerol esters, alkyl ethoxylates, and alkyl and alkyl phenol ethoxylates of all types, poly alkoxylated (e.g. ethoxylated or propoxylated) C₆-C₁₂ linear or branched alkyl phenols, C₆-C₂₂ linear or branched aliphatic primary or secondary alcohols, and C₂-C₈ linear or branched aliphatic glycols. Block or random copolymers of C₂-C₆ linear or branched alkylene oxides may also be suitable nonionic surfactants. Capped nonionic surfactants in which the terminal hydroxyl group is replaced by halide; C₁-C₈ linear, branched or cyclic aliphatic ether; C₁-C₈ linear, branched or cyclic aliphatic ester; phenyl, benzyl or C₁-C₄ alkyl aryl ether; or phenyl, benzyl or C₁-C₄ alkyl aryl ester may also be used. Sorbitan esters and ethoxylated sorbitan esters may also be useful nonionic surfactants. Other suitable nonionic surfactants may include mono or polyalkoxylated amides of the formula R¹CONR²R³ and amines of the formula R¹NR²R³ wherein R¹ is a C₅-C₃₁ linear or branched alkyl group and R² and R³ are C₁-C₄ alkyl, C₁-C₄ hydroxyalkyl, or alkoxylated with 1-3 moles of linear or branched alkylene oxides. Biosoft 91-6 (Stepan Co.) is an example of an alkyl ethoxylate (or alcohol ethoxylate) having a methylene chain length of C₉ to C₁₁ with an average of 6 moles of ethoxylation. An example of an alcohol ethoxylate is ECOSURF EH-9, which is more specifically an ethylene oxide-propylene oxide copolymer mono(2-ethylhexyl) ether, available from Sigma-Aldrich.

Alkylpolysaccharide nonionic surfactants are disclosed in U.S. Pat. No. 4,565,647 to Llenado, having a linear or branched alkyl, alkylphenyl, hydroxyalkyl, or hydroxyalkylphenyl group containing from about 6 to about 30 carbon atoms and a polysaccharide, e.g., a polyglycoside, hydrophilic group containing from about 1.3 to about 10 saccharide units. Suitable saccharides may include, but are not limited to, glucosides, galactosides, lactosides, and fructosides. Alkylpolyglycosides may have the formula: R²O(CnH₂ₙO)ₜ(glycosyl)ₓ wherein R² is selected from the group consisting of alkyl, alkylphenyl, hydroxyalkyl, hydroxyalkylphenyl, and mixtures thereof in which the alkyl groups contain from about 10 to about 18 carbon atoms; n is 2 or 3; t is from 0 to about 10, and x is from about 1.3 to about 10.

Fatty acid saccharide esters and alkoxylated fatty acid saccharide esters may also be suitable for use in the present invention. Examples include, but are not limited to, sucrose esters, such as sucrose cocoate, and sorbitan esters, such as polyoxyethylene(20) sorbitan monooleate and polyoxyethylene(20) sorbitan monolaurate.

Phosphate ester surfactants may also be suitable. These include mono, di, and tri esters of phosphoric acid with C₄-C₁₈ alkyl, aryl, alkylaryl, alkyl ether, aryl ether and alkylaryl ether alcohols (e.g. disodium octyl phosphate).

Zwitterionic surfactants may be suitable. As zwitterionic surfactants include both a positive and negative functional group, they may also be classified as nonionic surfactants. Many such zwitterionic surfactants contain nitrogen. Examples of such include amine oxides, sarcosinates, taurates and betaines. Examples include C₈-C₁₈ alkyldimethyl amine oxides (e.g., octyldimethylamine oxide, lauryldimethylamine oxide (also known as lauramine oxide), and cetyldimethylamine oxide), C₄-C₁₆ dialkylmethylamine oxides (e.g. didecylmethylamine oxide), C₈-C₁₈ alkyl morpholine oxide (e.g. laurylmorpholine oxide), tetra-alkyl diamine dioxides (e.g. tetramethyl hexanane diamine dioxide, lauryl trimethyl propane diamine dioxide), C₈-C₁₈ alkyl betaines (e.g. decylbetaine and cetylbetaine), C₈-C₁₈ acyl sarcosinates (e.g. sodium lauroylsarcosinate), C₈-C₁₈ acyl C₁-C₆ alkyl taurates (e.g. sodium cocoylmethyltaurate), C₈-C₁₈ alkyliminodipropionates (e.g. sodium lauryliminodipropionate), and combinations thereof. Lauryl dimethyl amine oxide (Ammonyx LO) myristyl dimethyl amine oxide (Ammonyx MO), decylamine oxide (Ammonyx DO) are examples of suitable zwitterionic surfactants, available from Stepan Co.

Non-limiting examples of anionic surfactants include alkyl sulfates (e.g., C₈-C₁₈ linear or branched alkyl sulfates such as sodium lauryl sulfate (SLS), and sodium tetradecylsulfate), linear alkylbenzene sulphonic acids or sulfonates (HLAS), alkyl sulfonates (e.g., C₆-C₁₈ linear or branched alkyl sulfonates such as sodium octane sulfonate and secondary alkane sulfonates, alkyl ethoxysulfates, fatty acids and fatty acid salts (e.g., C₆-C₁₆ fatty acid soaps such as sodium laurate), and alkyl amino acid derivatives. Other examples may include sulfate derivatives of alkyl ethoxylate propoxylates, alkyl ethoxylate sulfates, alpha olefin sulfonates, C₆-C₁₆ acyl isethionates (e.g. sodium cocoyl isethionate), C₆-C₁₈ alkyl, aryl, or alkylaryl ether sulfates, C₆-C₁₈ alkyl, aryl, or alkylaryl ether methylsulfonates, C₆-C₁₈ alkyl, aryl, or alkylaryl ether carboxylates, sulfonated alkyldiphenyloxides (e.g. sodium dodecyldiphenyloxide disulfonate), and the like.

More specific examples of nonionic and/or zwitterionic surfactants include lauryl dimethyl amine oxide (Ammonyx LO), also known as lauramine oxide, myristyl dimethyl amine oxide (Ammonyx MO), decylamine oxide (Ammonyx DO), other amine oxides, any betaines, linear alcohol ethoxylates, secondary alcohol ethoxylates, alcohol propoxylates, alkyl polyglucosides, and combinations thereof.

### D. Additional Adjuvants

The formulation may optionally include and/or be used in combination with one or more additional adjuncts. The adjuncts include, but are not limited to, fragrances or perfumes, waxes, dyes and/or colorants, solubilizing materials, stabilizers, thickeners, defoamers, hydrotropes, buffers, builders, lotions and/or mineral oils, enzymes, cloud point modifiers, and/or preservatives, and or chaotropic agents. In one embodiment, buffering and pH adjusting agents, when used, include, but are not limited to, organic acids, mineral acids, alkali metal and alkaline earth salts of citrate, silicate, metasilicate, polysilicate, borate, carbonate, carbamate, phosphate, polyphosphate, pyrophosphates, triphosphates, tetraphosphates, ammonia, hydroxide, monoethanolamine, monopropanolamine, diethanolamine, dipropanolamine, triethanolamine, and/or 2-amino-2methylpropanol.

A buffering agent can be a low molecular weight, organic or inorganic material used for maintaining the desired pH. For buffers that can be used, see McCutcheon's EMULSIFIERS AND DETERGENTS, North American Edition, 1997, McCutcheon Division, MC Publishing Company which is incorporated herein by reference. In yet another and/or alternative embodiment, solubilizing materials, when used, can include, but are not limited to, hydrotropes (e.g., water soluble salts of low molecular weight organic acids such as the sodium and/or potassium salts of xylene sulfonic acid).

In still another and/or alternative embodiment, thickeners, when used, include, but are not limited to, polyacrylic acid, xanthan gum, calcium carbonate, aluminum oxide, alginates, guar gum, methyl, ethyl, clays, and/or propylhydroxycelluloses. In yet another and/or alternative embodiment, defoamers, when used, include, but are not limited to, silicones, aminosilicones, silicone blends, and/or silicone/hydrocarbon blends. In still a further and/or alternative embodiment, preservatives, when used, include, but are not limited to, mildewstats or bacteriostats, methyl, ethyl and propyl parabens, short chain organic acids (e.g., acetic, lactic and/or glycolic acids), bisguanidine compounds (e.g., Dantagard and/or Glydant) and/or short chain alcohols (e.g., ethanol and/or IPA). In one aspect of this embodiment, the mildewstats or bacteriostats include, but are not limited to, mildewstats (including non-isothiazolone compounds) include Kathon GC, a 5-chloro-2-methyl-4-isothiazolin-3-one, Kathon ICP, a 2-methyl-4-isothiazolin-3-one, and a blend thereof, and Kathon 886, a 5-chloro-2-methyl-4-isothiazolin-3-one, all available from Rohm and Haas Company; Bronopol, a 2-bromo-2-nitropropane-1,3-diol, from Boots Company Ltd.; Proxel CRL, a propyl-p-hydroxybenzoate, from ICI PLC; Nipasol M, an o-phenyl-phenol, Na+ salt, from Nipa Laboratories Ltd.; Dowicide A, a 1,2-Benzoisothiazolin-3-one, from Dow Chemical Co.; and Irgasan DP 200, a 2,4,4′-trichloro-2-hydroxydiphenylether, from Ciba-Geigy A.G.

Fragrances may be included in an amount of at least about 0.025%, at least about 0.05%, up to about 1%, up to about 0.5%, or up to about 0.1% weight of the antimicrobial formulation. Advantageously, low levels of 1,2-hexanediol (4.25%) were shown to solubilize 0.05% fragrance, without the need for any further solvents to solubilize the fragrance. This allows the antimicrobial formulation to be void of ethanol and other lower mono-alcohols, often used as solvents, the elimination of which is helpful in reducing or eliminating volatile organic compounds.

In an embodiment, the formulation may include a majority of water, such as at least about 50%, at least about 55%, at least about 60%, at least about 65%, at least about 70%, at least about 75%, at least about 80%, at least about 85%, or at least about 90% water. Such water may be present in free, unbound form.

In an embodiment, the formulation has a relatively low viscosity, e.g., similar to that of water, in order to facilitate dispensing and formation of small airborne droplets. For example, the formulation may have a viscosity of up to about 1000 cps, up to about 500 cps, or up to about 100 cps, such as from about 1 cps to about 100 cps.

In some aspects, the antimicrobial formulation may exhibit at least a 2-log reduction, or at least a 3-log reduction against an airborne target microbe. The target microbe may be Staphylococcus aureus, Klebsiella pneumonia, Pseudomonas aeruginosa, phi6, MS2, or any of a wide variety of other pathogenic microbes or surrogate microbes. The antimicrobial formulation may exhibit such a log reduction against the airborne target microbe within a given exposure time frame, such as within 60 minutes, within 30 minutes, within 15 minutes, within 10 minutes, or within 5 minutes.

### III. Exemplary Air Sanitization Delivery

In an aspect of the invention, the antimicrobial formulation described above is delivered as a mist to the air to be sanitized. To effectively deliver the antimicrobial formulation, a number of technical parameters are taken into consideration such as temperature, relative humidity, flow rate, particle size, density, vapor pressure, and other potential parameters. For example, smaller particle size is often correlated with relatively lower flow rates in lower cost delivery methods. Due to this, there is a need to balance particle size and flow rate for desired efficacy. Advantageously, the present systems, methods and formulations do not require expensive delivery equipment in order to achieve sanitization or disinfection. For example, existing systems often require use of an expensive thermal fogger device in order to be effective. While such a device can of course be used to deliver the present formulations, such is not necessary, and sanitization or disinfection of a given air space can be achieved through simple inexpensive delivery tools, such as through a trigger sprayer, trigger mist device, etc.

A variety of devices may be used to deliver the antimicrobial formulation. For example, some embodiments may use a pump sprayer, a mister, a nebulizer, a vaporizer, a cold fogger, a thermal fogger, an ultrasonic dispensing device, an electrostatic sprayer, plug-in dispensing devices, or combinations thereof. Different devices may provide for different particle size ranges of the delivered antimicrobial formulation. Some devices, such as aerosols and pump sprayers, are low cost and easy to use. Other devices, such as foggers (particularly a thermal fogger), may have better performance (smaller average particle size), however they are typically bulky, expensive, and may require training or professional use.

In an embodiment, the total number of moles of lactic acid or other organic acid and optional delivery of 1,2-hexanediol delivered to the air may be within a desired range. The total number of moles per cubic meter may be at least about 0.05 mmol/m³, at least about 0.075 mmol/m³, at least about 0.1 mmol/m³, at least about 0.2 mmol/m³, at least about 0.3 mmol/m³, at least about 0.4 mmol/m³, at least about 0.5 mmol/m³, up to about 5 mmol/m³, up to about 4 mmol/m³, up to about 3 mmol/m³, up to about 2.5 mmol/m³, up to about 2 mmol/m³, or up to about 1 mmol/m³. The mass or molar concentration ratio of 1,2-hexanediol or other diol to lactic acid or other organic acid may be within the ranges noted herein (e.g., about 3:1 to about 1:3, greater than 1:1, about 1.2:1 to about 4:1, or about 1.5:1 to about 3.5:1 or about 1.5:1 to about 2.5:1). In an embodiment, lactic acid may dominate, e.g., with a ratio of lactic acid to diol of greater than 1, such as from about 1.1 to about 2:1, or from about 1:1 to about 1.5:1, or from about 1:1 to about 1.3:1 (e.g., about 1.1:1). Concentration values provided are calculated theoretically on the assumption that all chemistries are fully vaporized into the gaseous form. Such values will typically be an overestimation relative to empirically measured values.

Some embodiments may employ a stepwise delivery method (e.g., delivery of any diol, followed by delivery of the organic acid, or vice versa). For example, a first step may include the delivery of 1,2-hexanediol or other diol while the second step may include the delivery of lactic acid or other organic acid. An advantage of such a stepwise delivery method may include improved efficacy where the target microbe is an enveloped virus.

### IV. Examples

The antimicrobial formulation includes one or more organic acids and optionally one or more diols. To determine viable organic acids, a number of organic acids were screened. In an embodiment, viable organic acids may have: (1) a vapor pressure under standard conditions (e.g., 20° C. and 1 atmosphere) of at least 0.02 Pa, and less than 1000 Pa, less than 500 Pa, less than 300 Pa, less than 200 Pa, less than 100 Pa, less than 50 Pa, or less than 20 Pa; (2) be soluble in water (e.g., at least 10 g/100 g water); and/or (3) have an ECHA Inhalation Threshold for the General Population (or similar standard,) of greater than 10 mg/m³ i.e., essentially harmless to humans). Results of screened organic acids are shown in Table 1 where lactic acid was identified as meeting desired guidelines.

TABLE 1

| | | | | ECHA |
|---|---|---|---|---|
| | | | Solubility in | Inhalation |
| | | | water (g of | Threshold for |
| | | Vapor | compound in | General |
| | | Pressure | 100 g of water | Population |
| Category | Organic Acid | (Pa) | at ~20° C.) | (mg/m³) |
| alpha hydroxy | lactic acid | 10.00¹ | Miscible¹ | No hazard |
| acid | | | | identified¹ |
| | glycolic acid | 0.41¹ | 30¹ | 2.6³ |
| | 3- | N/A² | Very soluble¹ | N/A² |
| | hydroxypropionic | | | |
| | acid | | | |
| | 2-hydroxybutyric | N/A² | N/A¹ | N/A² |
| | acid | | | |
| | citric acid | 0.000002³ | 59.2¹ | No hazard |
| | | | | identified¹ |
| fatty acid | acetic acid | 2080³ | 60.29¹ | No hazard |
| | | | | identified (local |
| | | | | effects: DNEL |
| | | | | 25 mg/m³)¹ |
| | propionic acid | 390² | 100¹ | 18.3² |
| | butyric acid | 100² | Very soluble¹ | 9.15³ |
| | valeric acid | 100² | 3.75² | N/A³ |
| | hexanoic acid | 5.80¹ | 1.03² | 4.35³ |
| | heptanoic acid | 1.35¹ | 0.2² | 8.7³ |
| | octanoic acid | 0.49¹ | 0.068³ | No hazard |
| | | | | identified¹ |
| keto acid | levulinic acid | 0.374¹ | 79.13¹ | Insufficient data |
| | | | | available² |
| | pyruvic acid | 172² | 100¹ | N/A² |
| dicarboxylic | adipic acid | 9.70¹ | 23¹ | 13³ |
| acid | | | | |
| | malonic acid | 0.00049³ | 76.3¹ | 1.04³ |
| unsaturated | fumaric acid | 0.02² | 0.7² | 10.43³ |
| carboxylic acid | | | | |
| | maleic acid | 0.001³ | 47.88¹ | No hazard |
| | | | | identified¹ |
| | malic | <0.001³ | 64.7¹ | 9³ |
| | sorbic acid | 0.018² | 0.156² | 52.17¹ |
| aromatic acid | benzoic acid | 0.11¹ | 0.35² | 1.5³ |
| amino acid | glycine | <0.001³ | 24.99¹ | No hazard |
| | | | | identified¹ |

¹= acceptable
²= possibility
³= unacceptable

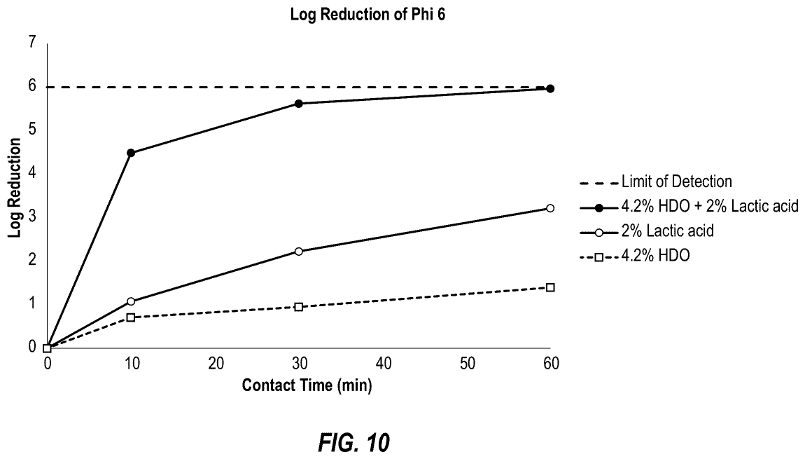

In addition to tested organic acids, a number of glycols, alcohols, diols and other candidates were also screened, for pairing with the organic acid. Viable candidates may also have: 1) a vapor pressure characteristics as noted above; 2) be soluble in water; and/or 3) have an ECHA Inhalation Threshold for the General Population of greater than 10 mg/m³. Additionally, a viable candidate should exhibit antimicrobial synergy with lactic acid or other selected organic acid when actually deployed together. Results of the candidate screening are shown in Table 2 where 1,2-hexanediol (HDO) was identified as meeting desired guidelines and was shown to have synergy with lactic acid. In particular, synergistic results are observed for combinations of 1,2-hexanediol and lactic acid, particularly where the mass or molar concentration ratio of 1,2-hexanediol to lactic acid may be within a range of 3:1 to 1:3. Further evidence of synergy is shown in FIG. 10.

While various glycols were screened, this data does not show any significant synergy provided when glycols are present. As such, while glycols may optionally be present, in an embodiment, the present formulations may be void, or substantially void of such glycol components (particularly propylene glycol and glycols including an ether group). The same may be said for various other candidate materials noted in Table 2.

TABLE 2

| | Vapor | ECHA Inhalation |
|---|---|---|
| | Pressure | Threshold for General |
| Candidate | (Pa) | Population (mg/m³) |
| triethylene glycol (TEG) | 0.066 | No hazard identified |
| propylene glycol (PG) | 20 | 50 |
| dipropylene glycol (DPG) | 1.3 | 70 |
| 1,2-hexanediol (HDO) | 0.576 | 30 |
| 1-dodecanol | 0.11 | 77 |
| methyl laurate | 0.55 | No hazard identified |
| 1,3-butanediol | 26 | No hazard identified |
| propyl acetate | 3300 | 149 |
| 1,2-butanediol | 10 | No hazard identified |

A promising combination was determined to include lactic acid (LA) as the organic acid and 1,2-hexanediol (HDO) as the diol in an antimicrobial formulation. A variety of experiments were performed to determine the efficacy of varying factors of an antimicrobial formulation which will now be discussed in further detail.

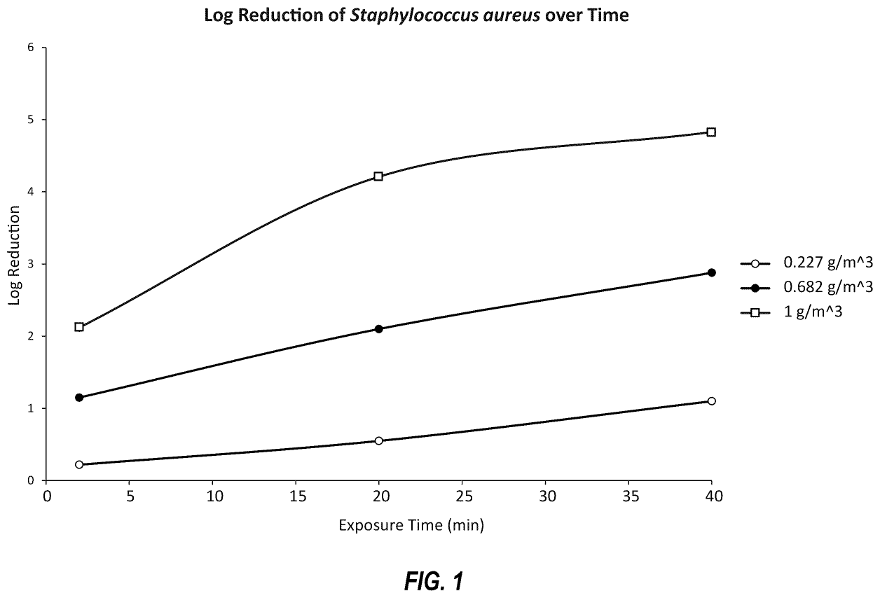

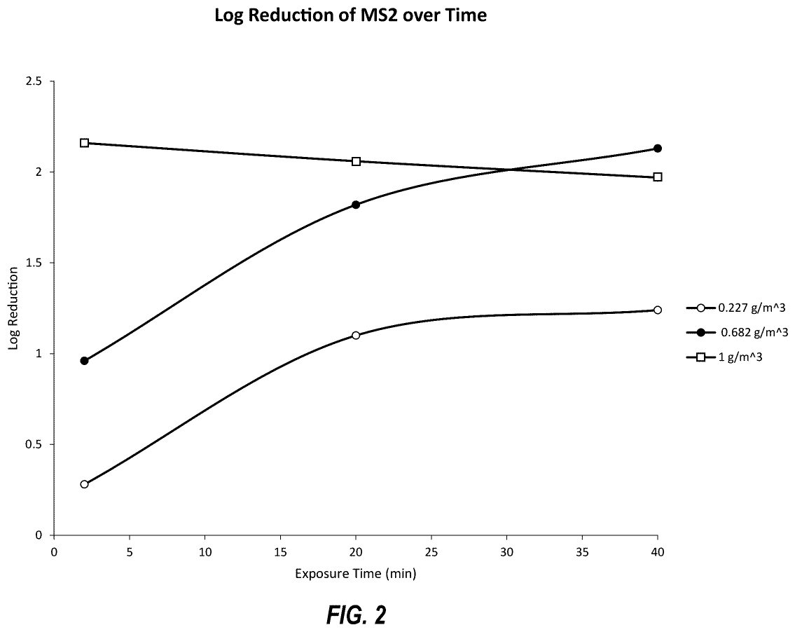

FIGS. 1 and 2 illustrate the log reduction relative to a baseline (untreated) control against different target microbes of an antimicrobial formulation which includes 2% lactic acid and 4.25% HDO. FIG. 1 shows the log reduction against Staphylococcus aureus over 40 minutes of exposure time. The antimicrobial formulation was delivered in different concentrations in an air chamber based on the number of squeezes delivered from a trigger spray device (e.g., such as available from FLAIROSOL). FIG. 1 illustrates that as dose increases (through more squeezes), the larger the log reduction against Staphylococcus aureus. After 40 minutes, the antimicrobial formulation provided about a 3-log reduction with 15 squeezes, providing a delivered formula concentration within the air being treated of 0.682 g/m³. With 22 squeezes, (and a delivered formula concentration of 1 g/m³), a 3-log reduction is achieved within about 10 minutes. While the 2% lactic acid and 4.25% HDO is one example, various other examples are of course also possible, some of which may perform even better. For example, another formulation may include about 3% lactic acid and about 2.5% HDO. Various examples are possible, within an HDO:LA ratio of about 3:1 to about 1:3.

FIG. 2 shows the log reduction relative to a baseline (untreated) control against MS2 over 40 minutes of exposure time. The antimicrobial formulation was delivered in different concentrations based on the number of squeezes delivered from a trigger sprayer of the same type used in FIG. 1. FIG. 2 further illustrates something of an efficacy plateau or diminishing return at about 15 squeezes (0.682 g/m³), as there is not a major difference with the 22 squeeze example (1 g/m³), after about 20 minutes.

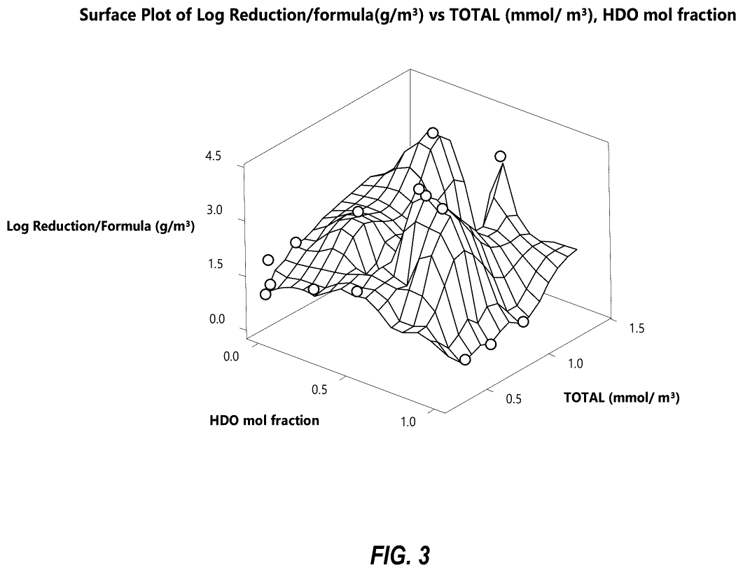

FIG. 3 illustrates a 3-axis plot, where one axis plots the total number of moles of the lactic acid/HDO antimicrobial formulation delivered, another axis plots the mol fraction of HDO in the delivered formulation, and the third axis plots the normalized log reduction, per g/m³ of formulation delivered into the air space being treated. In FIG. 3, the target microbe is phi 6. In this example, the antimicrobial formulation was delivered by a similar type trigger sprayer as in the previous examples, and a 10 minute exposure time was provided. FIG. 3 specifically shows how log reduction achieved varies with the total moles of antimicrobial formulation (mmol/m³) delivered, and the mol fraction of HDO relative to all HDO+LA in the formulation, and that synergistic combinations exist. Three levels are shown: a low-level concentration (0.254 mmol/m³), a medium level concentration (0.580 mmol/m³), and a high level concentration (1.164 mmol/m³). At low level concentrations, the efficacy level is driven by the fraction of lactic acid in the antimicrobial formulation. At a medium level concentration, significant efficacy is observed over a wide range of molar ratios of HDO to lactic acid (e.g., within a range of 1:3 to 3:1). As the molecular weights of lactic acid and HDO are similar (90 g/mol vs. 118 g/mol) to one another, molar ratios and mass ratios for the two components are also similar to one another. At high level concentrations, the efficacy of the antimicrobial formulation is improved at shorter exposure times but reached an efficacy plateau or point of diminishing returns at longer exposure times, particularly when the formulation included 25% to 75% HDO.

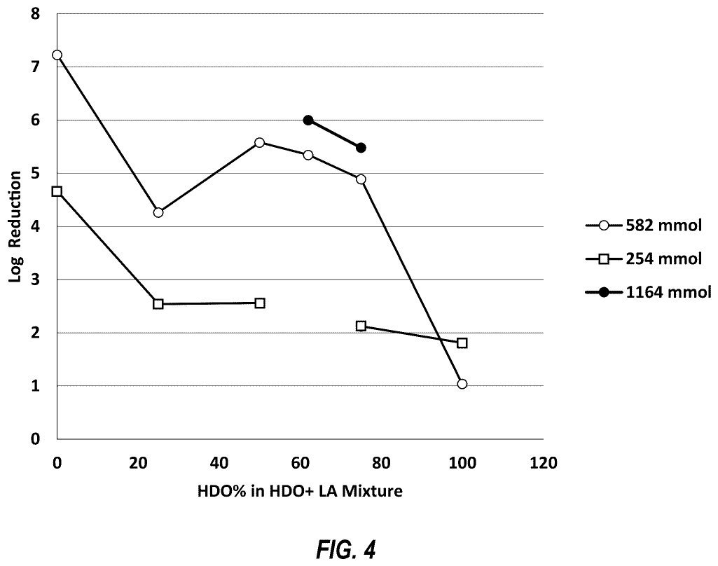

FIG. 4 illustrates data for log reduction against phi 6 versus percentage of HDO (HDO/HDO+LA) at different molar concentrations of total active (HDO+LA). FIG. 4 shows such data for a 60 minute exposure time. The efficacy plateaus or returns begin to diminish around 25% to 75% HDO in the antimicrobial formulation demonstrating that particular HDO+LA ratios provide synergy.

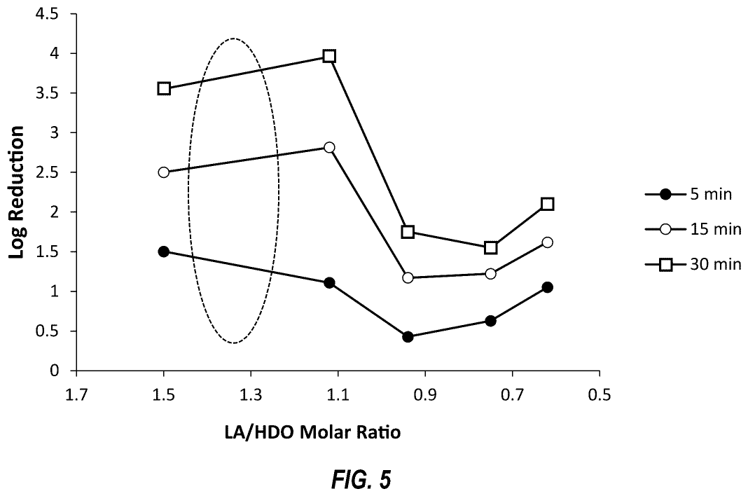

FIG. 5 illustrates data for log reduction against phi 6 versus molar ratio of lactic acid and 1,2-hexanediol. The log reduction was normalized against an untreated baseline. The concentration of 1,2-hexanediol stayed constant at 3.5% while the amount of lactic acid varied to give the desired molar ratios. Various exposure times (5 minutes, 15 minutes, and 30 minutes) were tested and the log reduction determined. A particularly beneficial ratio (dotted oval in FIG. 5) of particular synergy was observed around a 1.1 molar ratio of lactic acid to 1,2-hexanediol. This molar ratio illustrates a synergy between lactic acid and 1,2-hexanediol and unexpected results in a log reduction peak based on this particular ratio.

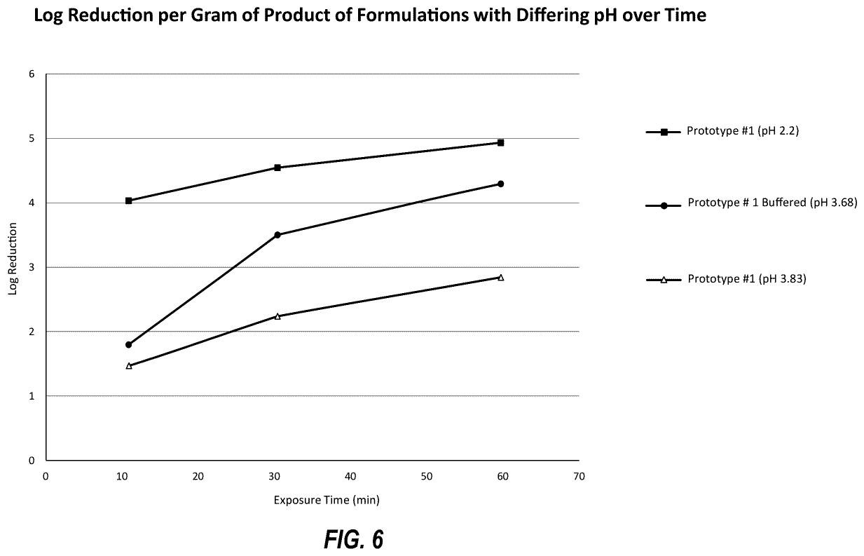

FIG. 6 illustrates the effect of varying pH values on log reduction against phi 6. One exemplary antimicrobial formulation containing 2% lactic acid and 4.25% HDO had a pH of 2.2. Another antimicrobial formulation containing an equal amount of lactic acid in acid form and 4.25% HDO after adjustment to a pH of 3.8 using NaOH and a third exemplary antimicrobial formulation with an equal amount of lactic acid in acid form, 4.25% HDO, and 2% citric acid (buffer) after adjustment to a pH of 3.7 with NaOH. The formulation with no buffer and a pH of 3.8 exhibited decreased antimicrobial efficacy, as compared to the formulation with a buffer, and a pH of 3.7. The formulation including the buffer showed a 5-log reduction after 60 minutes. In addition, it was found that reducing the amount of lactic acid and citric acid while staying in an optimal pH range also reduced the microefficacy.

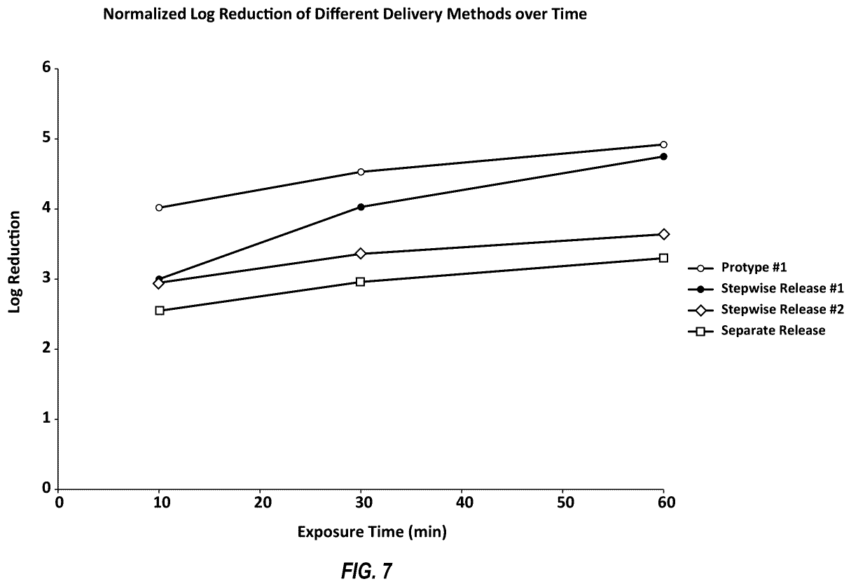

FIG. 7 illustrates normalized log reduction against phi 6 versus exposure time for various stepwise delivery methods of 2-part formulations including lactic acid and HDO. As tested, stepwise release #1 included release of HDO followed by lactic acid after 2 minutes. This example showed a 3-log reduction making it an effective delivery method. Delivery of a stepwise release can be accomplished in a number of ways. In some embodiments, lactic acid and HDO may be kept in two separate delivery chambers or delivery devices, where the user first releases one active ingredient and then subsequently releases the second active ingredient after a given period (e.g., within 60 minutes, within 30 minutes, within 10 minutes, within 5 minutes, such as within 1-5 minutes, or 2-3 minutes). In an embodiment, a programmable delivery device (e.g., a plug-in programmable dispensing device) may be programmed to provide a stepwise release.

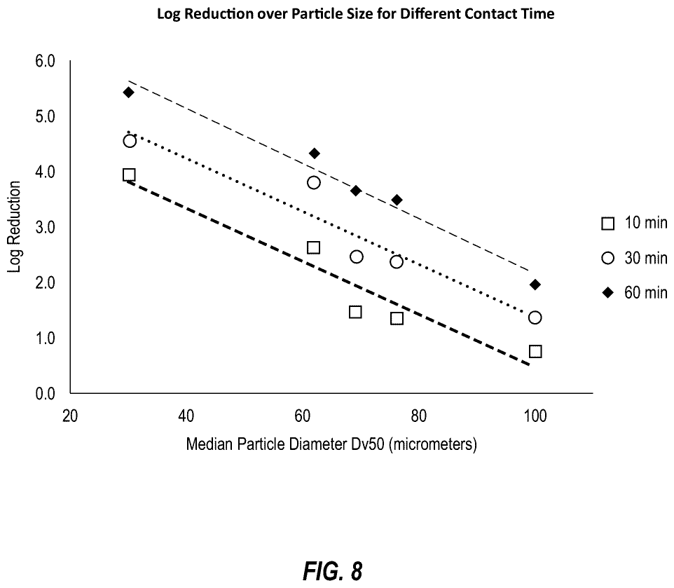

FIG. 8 illustrates log reduction against Staphylococcus aureus with a prototype formula delivered in different particle sizes at exposure times of 10 minutes, 30 minutes, and 60 minutes. The prototype #1 formulation (2% lactic acid, 4.25% 1,2-hexanediol) was delivered using a variety of spray devices which create different particle sizes. A 107 m³ chamber was used for experimentation and the chamber was dosed with 0.5 g/m³ of formulation. The log reduction was normalized against an untreated baseline. The median particle diameter (Dv50) for each spray was measured using laser diffraction. The laser diffraction measured droplet size at the center of the spray from 6 inches away. As is readily apparent from FIG. 8, smaller particle size provides for greater log reduction.

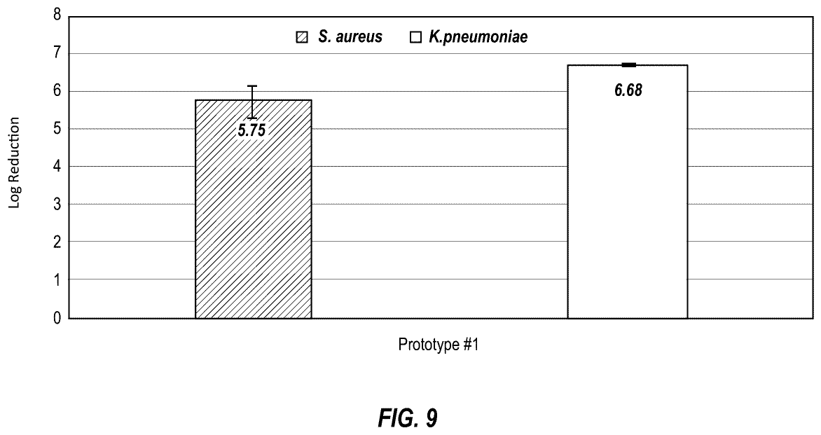

FIG. 9 illustrates antimicrobial efficacy on a soft surface (e.g., clothing or other fabric) using the prototype #1 formulation (2% lactic acid, 4.25% 1,2-hexanediol), against both Staphylococcus aureus and Klebsiella pneumoniae. The formulation was delivered to a 100% cotton surface with a 5 minute exposure time. The exemplary antimicrobial formulation exhibited a 5.76 log reduction for Staphylococcus aureus and a 6.68 log reduction for Klebsiella pneumoniae.

FIG. 10 charts the log reduction against airborne phi 6 for various formulas including 2% lactic acid alone, 4.2% 1,2-hexanediol alone, and a formula including both 4.2% 1,2-hexanediol and 2% lactic acid. The formulation including both the organic acid and the diol exhibits synergistic results, showing far improved log reduction values at all times between 10 minutes and 60 minutes. For example, while either lactic acid or 1,2-hexanediol alone only achieves about a 1 log reduction, at best, after 10 minutes, the combination of the two achieves a log reduction of about 4.5, orders of magnitude better than either component alone. Similar results are shown at 30 minute and 60 minute contact times, where the improvement is vast, compared to use of either component alone.

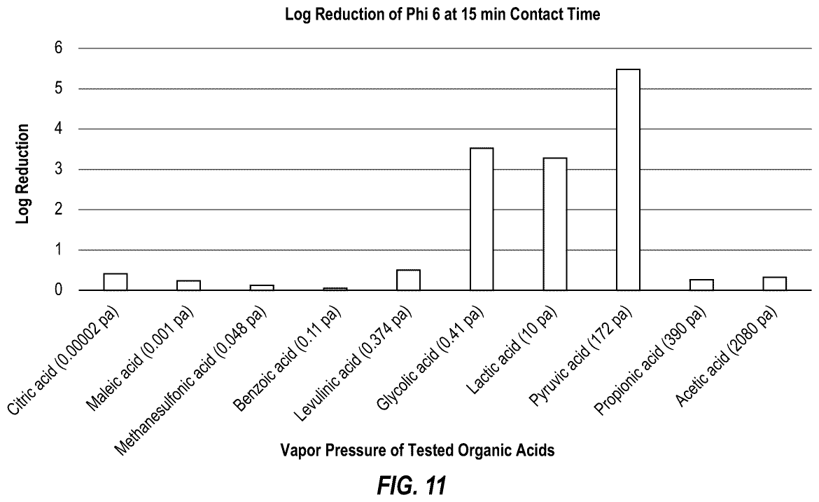

FIG. 11 charts the log reduction against airborne phi 6 for various organic acids, tested at various vapor pressures of such organic acid. Like the data shown in FIG. 10, testing was performed in a 1 m³ air chamber delivered via a sprayer, with a Dv50 of about 70 μm. For each test, 2 g of formula, including 222.2 mmol organic acid was delivered. The illustrated log reductions at 15 minutes contact time were charted against an untreated baseline. Glycolic acid, lactic acid, and pyruvic acid show good results (greater than a log 3 reduction), even without inclusion of a diol such as 1,2-hexanediol. As noted herein, glycolic acid may be less preferred than lactic acid or pyruvic acid, due to glycolic acid's ECHA inhalation threshold limit of 2.6 mg/m³. As shown in FIG. 11, a variety of organic acids show at least a 3-log reduction at vapor pressures from 0.10 Pa to 300 Pa, from 0.10 Pa to 200 Pa, from 0.40 Pa to 300 Pa, from 0.40 Pa to 200 Pa, from 0.41 Pa to 172 Pa, or from 10 Pa to 172 Pa.

In an embodiment, the selected organic acid has an aqueous solubility of at least about 10 g, at least about 20 g, at least about 24 g, at least about 24.99 g, such as from about 10 g to about 100 g, or about 10 g to about 50 g of the organic acid per 100 grams of water under standard conditions (room temperature and 1 atm). By way of example, lactic acid meets such requirements, glycolic acid has a solubility of about 30 g/100 g water, and pyruvic acid has a solubility of about 100 g/100 g of water.

Without departing from the spirit and scope of this invention, one of ordinary skill can make various modifications to the invention to adapt it to various usages and conditions. As such, these changes and modifications are properly, equitably, and intended to be, within the full range of equivalence of the following claims.

1. A system for air sanitization comprising:
a. an antimicrobial formulation comprising:
(i) one or more organic acids;
(ii) optionally, one or more diols; and
(a) wherein the organic acids have a vapor pressure in a range of 0.10 Pa to about 300 Pa at room temperature and standard pressure; and
b. a device for creating aerosolized droplets of the antimicrobial formulation wherein an average particle size is less than about 200 μm so that the formulation provides at least a 3-log reduction against airborne Staphylococcus aureus or another airborne target microbe within 60 minutes or less.

2. The system of claim 1, wherein the device for creating aerosolized droplets of the antimicrobial formulation provides an average particle size that is less than about 150 μm.

3. The system of claim 1, wherein the one or more organic acids comprise lactic acid.

4. The system of claim 1, wherein the formulation includes the one or more diols.

5. The system of claim 1, wherein the formulation includes the one or more diols, wherein the one or more diols comprise 1,2-hexanediol.

6. The system of claim 3, wherein the lactic acid is included in the formulation in an amount from about 0.1% to about 10%.

7. The system of claim 3, wherein the lactic acid is included in the formulation in an amount of about 2% to about 4%.

8. The system of claim 5, wherein the 1,2-hexanediol is included in the formulation in an amount from about 0.5% to about 10%.

9. The system of claim 3, wherein the formulation includes the one or more diols, wherein the one or more diols comprise 1,2-hexanediol, and a concentration ratio of the 1,2-hexanediol to the lactic acid in the formulation is from about 3:1 to about 1:3.

10. The system of claim 1, wherein a pH of the formulation is from about 2 to about 6.

11. The system of claim 1, further comprising a buffer.

12. The system of claim 11, wherein the buffer comprises citric acid.

13. The system of claim 12, wherein the citric acid is included in the formulation in an amount from about 0.1% to about 5%.

14. The system of claim 1, wherein the system provides at least a 3-log reduction against an airborne virus within 60 minutes or less.

15. The system of claim 1, wherein the device includes at least one of: a pump sprayer, a mister, a nebulizer, a vaporizer, a cold fogger, a thermal fogger, an ultrasonic dispensing device, or an electrostatic sprayer.

16. A system for air sanitization comprising:
a. an antimicrobial formulation comprising:
(i) one or more organic acids;
(ii) one or more diols; and
wherein the organic acids and diols have a vapor pressure of at least 0.02 Pa at room temperature and standard pressure; and
b. a device for creating aerosolized droplets of the antimicrobial formulation wherein an average particle size is less than about 200 μm so that the formulation provides at least a 3-log reduction against airborne Staphylococcus aureus or another airborne target microbe within 60 minutes or less.

17. A system for air sanitization comprising:
a. an antimicrobial formulation comprising:
(i) 0.1% to 5% by weight of at least one of lactic acid or citric acid;
(ii) 0.5% to 10% by weight of 1,2-hexanediol;
(iii) at least one of a fragrance or a surfactant;
wherein the formulation comprises at least 80% by weight water;
wherein a pH of the formulation is from about 2 to about 4; and
b. a device for creating aerosolized droplets of the antimicrobial formulation wherein the average particle size of such droplets is less than about 150 μm;
wherein the system provides at least a 3-log reduction against airborne Staphylococcus aureus within 60 minutes or less.

18. The system of claim 17, wherein the device includes at least one of a pump sprayer, a mister, a nebulizer, a vaporizer, a cold fogger, a thermal fogger, an ultrasonic dispensing device, or an electrostatic sprayer.

19. The system of claim 17, wherein the formulation provides at least a 3-log reduction against an airborne virus within 60 minutes or less.

20. The system of claim 17, wherein the formulation comprises the fragrance.
